(19) United States
(12) Patent Application Publication
(10) Pub. No.: US 2007/0250033 A1
(43) Pub. Date: Oct. 25, 2007

(54) SELF-ADHERING AND REUSABLE SURGICAL DRESSING

(76) Inventors: Georgina Holm, Fredericksburg, VA (US); Lindell L. Holm, Fredericksburg, VA (US)

Correspondence Address: HOLLAND & KNIGHT LLP, 10 ST. JAMES AVENUE, 11th Floor, BOSTON, MA, 02116-3889, US

(21) Appl. No.: 11/768,374

(22) Filed: Jun. 26, 2007

Related U.S. Application Data
(63) Continuation-in-part of application No. 29/233,429, filed on Jul. 5, 2005.

(51) Int. Cl. A61F 13/58

(52) U.S. Cl. 604/389

(57) ABSTRACT

A surgical dressing, and method of use, includes an absorbent pad having an inner surface, an outer surface, two long edges and two short edges configured to wrap around a user. Elastic material is attached to at least a portion of the two long edges and configured to stretch around the user when the absorbent pad is wrapped around the user. A first fastener is attached to the inner surface of the absorbent pad adjacent to one short edge and configured to attach to the outer surface of the absorbent pad to maintain the absorbent pad around the user.

### CROSS-REFERENCE TO RELATED APPLICATIONS

This application is a continuation-in-part of and claims the benefit under 35 U.S.C. 120 of U.S. Design patent application Ser. No. 29/233,429 filed Jul. 5, 2005, entitled “Self-Adhering and Reusable Surgical Dressing,” the entire contents of which is hereby incorporated by reference.

### FIELD OF INVENTION

The present invention relates to surgical dressings and more specifically to self-adhering and reusable surgical dressings.

### BACKGROUND

Ostomy surgery is a procedure in which an opening, called a stoma, is made in a person's body to enable waste product (i.e., stool or urine) to exit the body. The three types of ostomies are a colostomy, which is an opening in a colon or large intestine, an ileostomy, which is an opening into a part of a small intestine called the ileum, and a urostomy, which is an opening into a urinary system. The waste product of a colostomy and ileostomy is stool (bowl movements), while urine is the waste product of a urostomy. Ostomy surgery may be performed on children and adults alike.

After the surgery, the waste product is usually collected in a pouch system. Generally, the pouch system is held around the stoma by adhering to a ring to the skin around the stoma, to which the pouch is attached or attaches. As the waste product exits the stoma, it falls into the pouch. When the pouch is full, it is detached from the ring and emptied, or the ring is also detached and a new pouch system is adhered to the skin around the stoma. However, the adhesive used to attach the pouch to the skin may cause irritation or tear the skin when the pouch system is removed, particularly on infants, whose skin tends to be delicate and sensitive. Any irritation or tearing of the skin may lead to infections and other complications, and may be, at the least, uncomfortable or painful. Further, if the skin around the stoma is irritated or torn, the pouch system may leak.

Also, the pouch system hangs off the person's body. For children, the pouch system may interfere with normal activity, such as crawling for infants or play for older children. As the child becomes more active, he or she may pull the pouch off or various activities may cause the pouch to leak.

Further, positioning the pouch system over the stoma may require assistance, particularly for children and elderly people with limited dexterity. If the pouch system is not positioned and adhered properly, the pouch may leak.

### SUMMARY

The present invention provides a self-adhering and reusable surgical dressing which wraps around the person, covering the stoma.

In general, in one aspect, the invention features a surgical dressing including an absorbent pad having an inner surface, an outer surface, two long edges and two short edges configured to wrap around a user. Elastic material is attached to at least a portion of the two long edges and configured to stretch around the user when the absorbent pad is wrapped around the user. A first fastener is attached to the inner surface of the absorbent pad adjacent to one short edge and configured to attach to the outer surface of the absorbent pad to maintain the absorbent pad around the user.

In various embodiments, the absorbent pad is a washable absorbent fabric. For example, the washable absorbent fabric may be a cotton fabric. In other embodiments, the absorbent pad is a disposable absorbent material. For example, the disposable absorbent material may be a synthetic polymer.

In embodiments, the surgical dressing also includes a second fastener attached to the outer surface of the absorbent pad adjacent to the other short edge. The second fastener is configured to mate with the first fastener to maintain the absorbent pad around the user. In various embodiments, the first fastener and second fastener may be a hook and loop fastener, a button, a snap or adhesive tape.

In various embodiments, the absorbent pad may include various layers of material, and may also include different materials in various layers. For example, the outer surface of the absorbent pad may be a non-permeable layer.

In general, in another aspect, the invention features a method of collecting waste product from a stoma including wrapping an absorbent pad having an inner surface, an outer surface, two long edges and two short edges around a user such that one long edge is above the stoma and the other long edge is below the stoma. A first fastener that is attached to the inner surface of the absorbent pad adjacent to one short edge is connected to the outer surface of the absorbent pad to maintain the absorbent pad around the user.

In various embodiments, the absorbent pad also includes an elastic material attached to at least a portion of the two long edges and is configured to stretch around a user when the absorbent pad is wrapped around the user.

In embodiments, the absorbent pad also includes a second fastener attached to the outer surface of the absorbent pad adjacent to the other short edge. The second fastener is configured to mate with the first fastener to maintain the absorbent pad around the user. In various embodiments, the first fastener and second fastener may be a hook and loop fastener, a button, a snap or adhesive tape.

In various embodiments, the absorbent pad may be a washable absorbent fabric. For example, the washable absorbent fabric may be a cotton fabric. In other embodiments, the absorbent pad may be a disposable absorbent material. For example, the disposable absorbent material may be a synthetic polymer.

The invention can be implemented to realize one or more of the following advantages. The surgical dressing does not include any adhesive or adhere to the skin, so it cannot tear the skin and is less likely to irritate the skin around the stoma. Because the surgical dressing does not need to adhere to the skin around the stoma, there is less likelihood of any leakage.

The surgical dressing does not hang off the body, so there is less chance that activity will cause the surgical dressing to become detached or leak. Further, because the surgical dressing is wrapped around the person, it is less likely to interfere with any activities.

Also, the surgical dressing is easier to apply. It does not require precise positioning, thus a child or elderly person with limited dexterity may be able to change the surgical dressings by themselves. Further, the fasteners enable periodic removal of the surgical dressing to check and attend to the stoma, especially immediately after surgery.

Further, the surgical dressing absorbs the waste product and moves it away from the stoma. The surgical dressing may be made of a breathable material that enables some air movement around the skin. Unlike a pouch system, with which occasional gas pressure release is necessary, the breathable fabric of the surgical dressing prevents gas buildup. Also, when used in conjunction with a pouch system, it may enable the skin around the stoma to heal in the event of a tear or irritation caused by the pouch system.

Further still, the surgical dressing is more cost effective than a pouch system.

Other features and advantages of the invention are apparent from the following description, and from the claims.

### BRIEF DESCRIPTION OF THE DRAWINGS

FIG. 1 is a front elevation view of a self-adhering and reusable surgical dressing.

FIG. 2 is a rear elevation view of the self-adhering and reusable surgical dressing of FIG. 1.

FIG. 3 is a side elevation view of the self-adhering and reusable surgical dressing of FIG. 1.

FIG. 4 is a top plan view of the self-adhering and reusable surgical dressing of FIG. 1.

Like reference numbers and designations in the various drawings indicate like elements.

### DETAILED DESCRIPTION

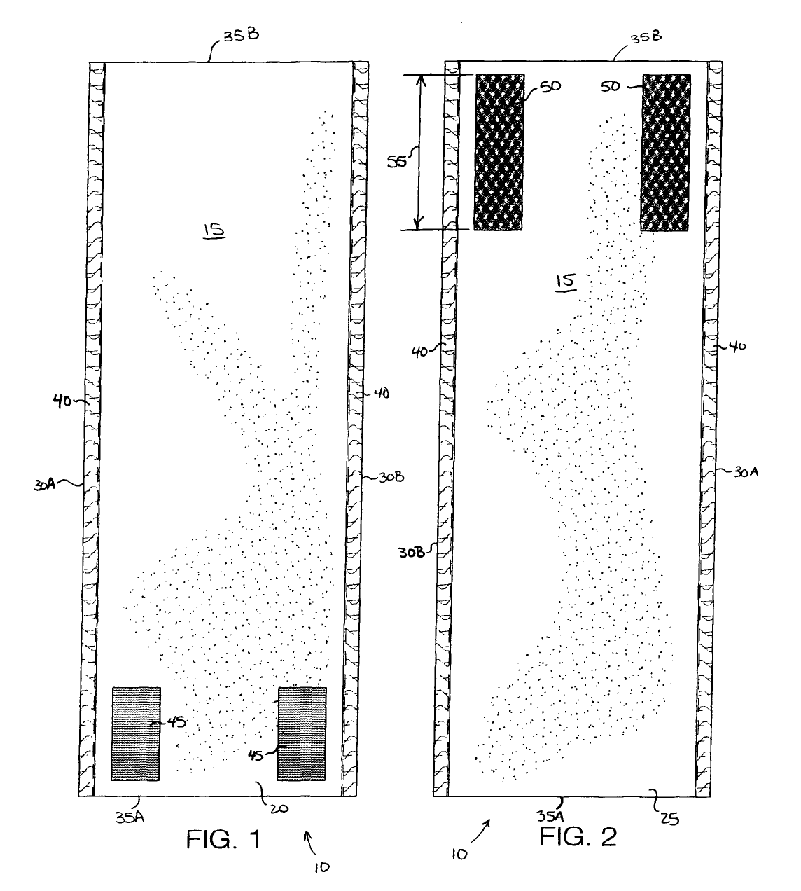

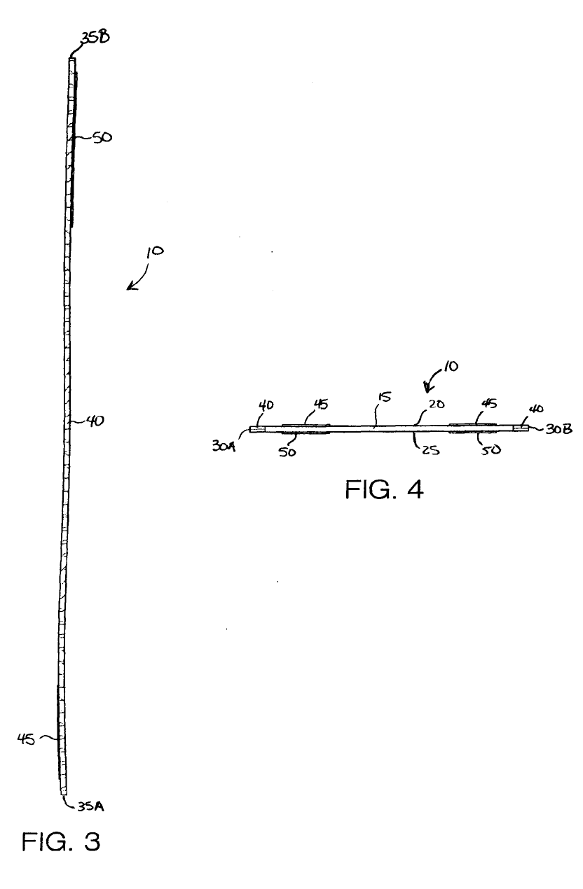

As shown in FIGS. 1-4, a surgical dressing 10 includes an absorbent pad 15 having an inner surface 20 and an outer surface 25. The inner surface 20 faces a user when worn. The absorbent pad 15 is generally rectangular in shape and includes long edges 30A, 30B and short edges 35A, 35B.

Each long edge 30A, 30B includes elastic material 40 that runs at least a portion of a length of the long edges 30A, 30B. The elastic material 40 may be an elastic band that is sewn into the absorbent pad 15, or attached to the absorbent pad 15 with an adhesive. The inner surface 20 includes a fastener 45 adjacent to one short edge 35A, and the outer surface 25 includes a second fastener 50 adjacent to the other short edge 35B configured to enable the fastener 45 to overlap the second fastener 50 in use, as described below. The fasteners 45, 50 may be mating components of a hook and loop fastener, such as Velcro®.

In use, the surgical dressing 10 is wrapped around a user with the inner surface 20 facing the user and covering the stoma and the long edges 30A, 30B encircling the user above and below the stoma. The fastener 45 is mated to the second fastener 50 to maintain the surgical dressing 10 around the user by overlapping the fasteners 45, 50. The fasteners 45, 50 are configured to enable variable adjustment to provide the best fit of the absorbent pad 15 around the user. For example, the fastener 45 may be a hook portion of the hook and loop fastener extending the length of the short edge 35A, while the second fastener 50 may be a length 55 of the loop portion of the hook and loop fastener running parallel to the long edges 30A, 30B of the absorbent pad 15. This enables the fastener 45 to be positioned anywhere along the length 55 running parallel to the long edges 30A, 30B of the absorbent pad 15.

The absorbent pad 15 may be sized to fit any user. For example, for use with infants, the absorbent pad 15 may be approximately 26 inches long and 7.5 inches wide. The elastic material 40 may be 16 inches long, and stretched to 26 inches when it is attached to the absorbent pad 15. Attaching the elastic material 40 to the absorbent pad 15 while it is stretched will cause the long edges 30A, 30B of the absorbent pad 15 to contract around the user when worn. The elastic material 40 along the long edges 30A, 30B maintain contact between the absorbent pad 15 and the user to minimize any potential gaps at the long edges 30A, 30B. Further, the elastic material 40 may be configured to enable the absorbent pad 15 to fit loosely or be spaced away from the user so that it does not continually rub against the user's skin and, more importantly, the stoma.

The absorbent pad 15 may be made of an absorbent material. For example, the absorbent pad 15 may be made of multiple layers of an absorbent cotton fabric, such as 8 layers of birdseye cloth, which may be washed and reused. However, other absorbent cotton fabrics may be used, such as terry cloth or flannel, and any number of layers may be used. The absorbent pad 15 should be made such that it will retain and absorb any urine or stool that is excreted from the stoma, and may be a woven, non-woven or knit fabric.

It is to be understood that the foregoing description is intended to illustrate and not to limit the scope of the invention, which is defined by the scope of the appended claims. Other embodiments are within the scope of the following claims. For example, while the absorbent pad 15 has been described as multiple layers of absorbent cotton fabric, the absorbent pad 15 may be made of any absorbent, washable fabric. Further, the absorbent pad 15 may be made of a combination of fabrics and/or materials. For example, the inner surface 20 may be made of absorbable cotton fabric while the outer surface is made of a non-absorbable, barrier layer such as plastic or nylon, to further prevent any leaking and retain odors. Further still, the absorbent pad 15 may be made of a disposable, non-washable material, such as disposable diaper material.

Also, while the size of the absorbent pad 15 has been described for an infant, any size may be used to provide various sized surgical dressings 10. For example, for an adult, the absorbent pad 15 may be 43 inches long by 9 inches wide.

Further, while the fasteners 45, 50 have been described as a hook and loop fastener, the fasteners 45, 50 may be any suitable fastener system. For example, the fasteners 45, 50 may be mating halves of snaps or buttons. Further still, fastener 45 may be an adhesive strip that adheres directly to the outer surface 25 of the absorbent pad 15. In that embodiment, the second fastener 45 may be eliminated. Also, while the fasteners 45, 50 have been shown in a particular configuration (i.e., parallel to the long edges), other configurations are possible. For example, the fastener 45 may be a single strip of hook material parallel to the short edge 35A while the second faster 50 may be two strips of loop material parallel to the long edges 30A, 30B.

Also, while the elastic material 40 has been shown as attached to the entire length of the long edges 30A, 30B, the elastic material 40 may be attached to only a portion of the length of the long edges 30A, 30B.

What is claimed is:

1. A surgical dressing comprising:
an absorbent pad having an inner surface, an outer surface, two long edges and two short edges configured to wrap around a user;
elastic material attached to at least a portion of the two long edges configured to stretch around the user when the absorbent pad is wrapped around the user; and
a first fastener attached to the inner surface of the absorbent pad adjacent to one short edge configured to attach to the outer surface of the absorbent pad to maintain the absorbent pad around the user.

2. The surgical dressing of claim 1 wherein the absorbent pad is a washable absorbent fabric.

3. The surgical dressing of claim 2 wherein the washable absorbent fabric is a cotton fabric.

4. The surgical dressing of claim 1 wherein the absorbent pad is a disposable absorbent material.

5. The surgical dressing of claim 4 wherein the disposable absorbent material is a synthetic polymer.

6. The surgical dressing of claim 1 further comprising a second fastener attached to the outer surface of the absorbent pad adjacent to the other short edge and configured to mate with the first fastener to maintain the absorbent pad around the user.

7. The surgical dressing of claim 6 wherein the first fastener and second fastener are selected from the group consisting of a hook and loop fastener, a button, a snap and adhesive tape.

8. The surgical dressing of claim 1 wherein the outer surface further includes a non-permeable layer.

9. A method of collecting waste product from a stoma comprising:
wrapping an absorbent pad having an inner surface, an outer surface, two long edges and two short edges around a user such that one long edge is above the stoma and the other long edge is below the stoma; and
connecting a first fastener attached to the inner surface of the absorbent pad adjacent to one short edge to the outer surface of the absorbent pad to maintain the absorbent pad around the user.

10. The method of claim 9 wherein the absorbent pad further includes an elastic material attached to at least a portion of the two long edges configured to stretch around a user when the absorbent pad is wrapped around the user.

11. The method of claim 9 wherein the absorbent pad further comprises a second fastener attached to the outer surface of the absorbent pad adjacent to the other short edge and configured to mate with the first fastener to maintain the absorbent pad around the user.

12. The method of claim 11 wherein the first fastener and second fastener are selected from the group consisting of a hook and loop fastener, a button, a snap and adhesive tape.

13. The surgical dressing of claim 9 wherein the absorbent pad is a washable absorbent fabric.

14. The surgical dressing of claim 13 wherein the washable absorbent fabric is a cotton fabric.

15. The surgical dressing of claim 9 wherein the absorbent pad is a disposable absorbent material.

16. The surgical dressing of claim 15 wherein the disposable absorbent material is a synthetic polymer.

17. A surgical dressing comprising:
an absorbent pad having an inner surface, an outer surface, a first long edge and a second long edge, and a first short edge and a second short edge, configured to wrap around a user;
a first elastic band attached to at least a portion of the first long edge and a second elastic band attached to at least a portion of the second long edge, the first elastic band and the second elastic band configured to stretch around the user when the absorbent pad is wrapped around the user;
a first fastener attached to the inner surface of the absorbent pad adjacent to the first short edge; and
a second fastener attached to the outer surface of the absorbent pad adjacent to the second short edge and configured to mate with the first fastener to maintain the absorbent pad around the user.

18. The surgical dressing of claim 17 wherein the first fastener and second fastener are selected from the group consisting of a hook and loop fastener, a button, a snap and adhesive tape.

19. The surgical dressing of claim 17 wherein the absorbent pad is a washable absorbent cotton fabric.

20. The surgical dressing of claim 17 wherein the absorbent pad is a disposable absorbent material.
